(19) United States
(12) Patent Application Publication
(10) Pub. No.: US 2024/0374878 A1
(43) Pub. Date: Nov. 14, 2024

(54) Scraper Balloon Catheter Device

(71) Applicant: NOVOSERT LTD., Nesher (IL)

(72) Inventors: Avraham SHEKALIM, Nesher (IL); Noam PELEG, Gan Ner (IL)

(21) Appl. No.: 18/424,964

(22) Filed: Jan. 29, 2024

Related U.S. Application Data
(60) Provisional application No. 63/465,268, filed on May 10, 2023.

(51) Int. Cl. A61M 25/10; A61M 25/01

(52) U.S. Cl. CPC A61M 25/104; A61M 2025/0177; A61M 2025/1056; A61M 2025/109

(57) ABSTRACT

An angioplasty enhancement device for use with an angioplasty system including a guiding catheter, a guidewire and a balloon catheter includes an elongated shaft terminating in a guide extension catheter. A loop of flexible material is displaceable between a proximal position located within a lumen of the guide extension catheter and a distal position extending beyond the guide extension catheter for overlying an external surface of the balloon catheter. A displaceable element extending along the elongated shaft controls displacement of the loop. When the balloon catheter is inserted along the guidewire in a deflated state so as to extend across a lesion, pushing the displaceable element is effective to advance the loop from the proximal position over the external surface of the balloon catheter to the distal position so that subsequent inflation of the balloon catheter presses the loop against the lesion.

### FIELD AND BACKGROUND OF THE INVENTION

The present invention relates to intravascular catheters and, in particular, a device for percutaneous deployment to remove material from an internal surface of a blood vessel, and corresponding methods.

Fatty and calcified deposits accumulated in the coronary or peripheral arteries of a human patient seriously threaten the health of the patient. Various percutaneous coronary intervention (PCI) treatments are currently available to open a narrowed or blocked segment of a blood vessel. One such treatment is balloon angioplasty, in which a balloon catheter is inserted into the body, manipulated to the location where the blockage appears to be, and inflated to expand the lumen and thereby to increase blood flow. A stent may be applied inside the blood vessel to provide support for the vessel in its expanded condition.

Advanced age, renal disease and diabetes have all been associated with coronary artery calcification (CAC). Severe CAC affecting between 6 to 20% of patients treated with percutaneous coronary intervention (PCI). PCI for heavily calcified lesions is associated with difficulty in adequately dilating the artery; inability to deliver and implant stents appropriately; greater risk of acute complications; and higher restenosis rates. Calcified coronary lesions can pose special problems and may prevent stent delivery or expansion, and increase the likelihood of stent thrombosis and/or restenosis.

Calcified lesions may form a particular threat to Drug Eluting Stents (DES), as damage to the polymer/drug coating and inadequate diffusion of the drug that may decrease DES effectiveness. Hence, before stenting, it is important to achieve preparation and modification of heavily calcified lesions.

Recent evidence indicates that modification of the calcified lesions may play a pivotal role in enhancing the clinical outcomes of angioplasty. Vessel preparation has shifted from a trend to a consistent element of treatment algorithms. Surgical techniques and tools have been developed for this purpose. Examples for these tools are cutting balloons and scoring balloons. These tools essentially combine the features of a conventional balloon with wires mounted on the surface, respectively. These wires function as microsurgical blades that give these balloons their cutting or scoring properties. Cutting balloons and scoring balloons are well suited for treating fibrous and calcified lesions that are resistant to dilation.

An example for these devices is described in US Application 2005/0021071A1, published Jan. 27, 2005, in which a scoring structure, for example, in the form of a separate expandable cage, is carried by an inflatable balloon so as to score the stenotic material when expanded by the balloon in the blood vessel.

The major limitation of cutting/scoring balloons devices is high crossing profile and greater rigidity that cause problems in crossing calcified lesions. In addition, some other procedural complications, such as: distal embolization, perforations, dissections, and tissue injury that may cause late restenosis, are also associated with cutting balloons and scoring balloons.

Another treatment in use is an atherectomy procedure, which involves the removal of the atheroma (e.g., deposits or degenerative accumulations) from the affected vessel with a cutting device delivered to the treatment site by a catheter. The known atherectomy treatments, however, are subject to a number of serious risks, including the possibility of a heart attack during the procedure, a closing of the artery necessitating emergency bypass surgery, bleeding caused by damage to the vessel walls, and irregular heart rhythms caused by the trauma to the body. In addition, an atherectomy treatment is very costly and can also lead to early complications.

### SUMMARY OF THE INVENTION

The present invention is a device for percutaneous deployment to remove material from an internal surface of a blood vessel, and corresponding methods.

According to the teachings of an embodiment of the present invention there is provided, an angioplasty enhancement device for use with an angioplasty system including a guiding catheter, a guidewire inserted through the guiding catheter and extending beyond the guiding catheter to cross a lesion, and a balloon catheter insertable along the guidewire through the guiding catheter to reach the lesion, the angioplasty enhancement device comprising: (a) an elongated shaft sized for insertion through the guiding catheter and to extend beyond the guiding catheter, at least a distal portion of the elongated shaft being formed as a catheter with at least one hollow lumen to accommodate the guidewire and the balloon catheter; (b) a loop of flexible material displaceable between a proximal position located within the lumen of the elongated shaft and a distal position extending beyond the elongated shaft for overlying an external surface of the balloon catheter; and (c) a displaceable element extending along the elongated shaft and associated with at least one side of the loop so that displacement of a part of the displaceable element from a proximal end of the guiding catheter controls displacement of the loop between the proximal position and the distal position, such that, when the balloon catheter is inserted along the guidewire in a deflated state so as to extend across the lesion, pushing the displaceable element is effective to advance the loop from the proximal position over the external surface of the balloon catheter to the distal position so that subsequent inflation of the balloon catheter is effective to press the loop against the lesion.

According to a further feature of an embodiment of the present invention, the distal portion of the elongated shaft is implemented as a guide extension catheter, and wherein a majority of a length of the elongated shaft proximal to the guide extension catheter is implemented as a tube sized for accommodating only the displaceable element while lying in side-by-side relation to the guidewire and the balloon catheter within the guiding catheter.

According to a further feature of an embodiment of the present invention, the loop is a wire loop.

According to a further feature of an embodiment of the present invention, the loop is one of at least two loops spaced apart so as to advance around at least two regions of a periphery of the balloon catheter.

According to a further feature of an embodiment of the present invention, there is also provided a handle associated with a proximal end of the elongated shaft, and wherein displacement of the loop is controlled by an actuator associated with the handle.

There is also provided according to the teachings of an embodiment of the present invention, an angioplasty device for percutaneous transluminal deployment to treat a lesion within a blood vessel of a human, the device comprising: (a) a deployment catheter for percutaneous transluminal deployment within the blood vessel; (b) an inflatable balloon configured for deployment from the catheter; (c) and a displaceable loop of flexible material displaceable relative to the balloon between a proximal position and a distal position, the distal position extending across an external surface of the balloon, at least one side of the loop being connected via a displaceable element extending along the catheter so that displacement of a part of the displaceable element outside the body controls displacement of the loop relative to the balloon, wherein, when the balloon is inserted in a deflated state so as to extend across the lesion, pushing the at least one side of the loop is effective to advance the loop from the proximal position over the external surface of the balloon to the distal position, and wherein subsequent inflation of the balloon is effective to press the loop against the lesion.

According to a further feature of an embodiment of the present invention, the loop of flexible material is a wire loop.

According to a further feature of an embodiment of the present invention, the loop is associated with the displaceable element so that displacement of the displaceable element displaces the entirety of the loop relative to the balloon.

According to a further feature of an embodiment of the present invention, one end of the loop is deployed in a fixed position relative to the deployment catheter.

According to a further feature of an embodiment of the present invention, the loop is one of at least two displaceable loops spaced around a periphery of the balloon.

According to a further feature of an embodiment of the present invention, the deployment catheter comprises a handle, and wherein displacement of the loop is controlled by an actuator associated with the handle.

According to a further feature of an embodiment of the present invention, the deployment catheter comprises a lumen configured for interchangeable insertion of the inflatable balloon.

There is also provided according to the teachings of an embodiment of the present invention, an angioplasty device for percutaneous transluminal deployment to treat a lesion within a blood vessel of a human, the device comprising: (a) an elongated flexible shaft, having a plurality of internal lumens, for percutaneous transluminal deployment within the blood vessel; (b) an inflatable balloon associated with a distal end of the shaft, an interior of the inflatable balloon being in fluid communication with a first lumen of the plurality of lumens for selectively inflating and deflating the inflatable balloon; (c) and a loop of flexible material retractable to a proximal position in a second lumen of the plurality of lumens and selectively displaceable so as to extend across an external surface of the balloon in a distal position, at least one side of the loop being connected via a displaceable element extending along the second lumen so as to be displaceable from outside the body, wherein, when the balloon is inserted in a deflated state so as to extend across the lesion, pushing the at least one side of the loop is effective to advance the loop from the proximal position over the external surface of the balloon to the distal position, and wherein subsequent inflation of the balloon is effective to press the loop against the lesion.

According to a further feature of an embodiment of the present invention, the loop of flexible material is a wire loop.

According to a further feature of an embodiment of the present invention, the plurality of lumens further includes an aspiration lumen for aspirating the material scraped from the internal surface of the blood vessel from adjacent to the proximal end of the balloon.

According to a further feature of an embodiment of the present invention, the loop is associated with the displaceable element so that displacement of the displaceable element displaces the entirety of the loop relative to the balloon.

According to a further feature of an embodiment of the present invention, one end of the loop is deployed in a fixed position relative to the elongated shaft.

According to a further feature of an embodiment of the present invention, the loop is one of at least two displaceable loops spaced around a periphery of the balloon.

According to a further feature of an embodiment of the present invention, there is also provided an actuator associated with a proximal end of the elongated shaft and configured to control displacement of the loop.

### BRIEF DESCRIPTION OF THE DRAWINGS

The invention is herein described, by way of example only, with reference to the accompanying drawings, wherein:

FIG. 1 is a schematic isometric view of an angioplasty enhancement device, constructed and operative according to an embodiment of the present invention, in use with an angioplasty system including a guiding catheter, a guidewire and a balloon catheter;

FIG. 2A is a schematic representation of a configuration of a conventional angioplasty system with which the angioplasty enhancement device of the present invention may be used;

FIG. 2B is a schematic representation of the angioplasty enhancement device of FIG. 1 integrated into the configuration of FIG. 2A;

FIGS. 3A and 3B are schematic partial isometric views of a displaceable flexible loop shown in a distal position, overlapping a balloon of a balloon catheter, and a proximal position, withdrawn from the balloon;

FIGS. 4A-4F are isometric cut-away views illustrating a sequence of states of the balloon catheter and the displaceable flexible loop during an angioplasty procedure according to an implementation of the present invention;

FIGS. 5A-5E are side cut-away views illustrating a sequence of states of the balloon catheter and the displaceable flexible loop during an angioplasty procedure according to an implementation of the present invention;

FIGS. 6A-6C are schematic views of an alternative implementation of the displaceable flexible loop illustrated together with the balloon in states similar to FIGS. 4B-4D, respectively;

FIGS. 7A and 7B are isometric views of a double-loop structure for use in a variant implementation of the present invention, shown in a compressed form and in a radially-expanded form, respectively;

FIGS. 8A-8C are views similar to FIGS. 6A-6C employing the double-loop structure of FIGS. 7A and 7B;

FIG. 9A is an axial cross-sectional view taken through a handle from the device of FIG. 1;

FIG. 9B is an enlarged view of the region of FIG. 9A designated R1;

FIG. 10A is a side view of the handle of FIG. 9A;

FIGS. 10B and 10C are cross-sectional views taken along the lines X1-X1 and X2-X2, respectively, in FIG. 10A;

FIG. 10D is an enlarged view of the region R2 of FIG. 10C;

FIG. 11 is a schematic representation of an axial cross-section illustrating an implementation of the device of FIG. 1 during deployment within a blood vessel;

FIGS. 12A and 12B are schematic cross-sectional views taken along the line X3-X3 in FIG. 11 when implemented with a rapid exchange (Rx) balloon catheter and an over-the-wire (OTW) balloon catheter, respectively;

FIG. 13 is a schematic representation of an axial cross-section illustrating an alternative implementation of the device of FIG. 1 during deployment within a blood vessel;

FIGS. 14A and 14B are schematic cross-sectional views taken along the line X4-X4 in FIG. 13 when implemented with an Rx balloon catheter and an OTW balloon catheter, respectively;

FIG. 15 is a schematic representation of an axial cross-section illustrating a further alternative implementation of the device of FIG. 1 during deployment within a blood vessel;

FIGS. 16A and 16B are schematic cross-sectional views taken along the line X5-X5 in FIG. 15 when implemented as an Rx device and an OTW device, respectively;

FIG. 17A is a schematic isometric view of an angioplasty enhancement device, constructed and operative according to a further embodiment of the present invention, implemented as a guide extension catheter for use with an angioplasty system including a guiding catheter, a guidewire and a balloon catheter;

FIGS. 17B-17D are cross-sectional views taken through FIG. 17A long lines X6-X6, X7-X7 and X8-X8, respectively;

FIG. 18A is an enlarged partial axial cross-sectional view of a distal part of the device of FIG. 17A;

FIGS. 18B-18D are enlarged views of the regions of FIG. 18A designated as R3, R4 and R5, respectively;

FIGS. 19A and 19B are side and top views, respectively, of a connector, visible in FIG. 18B;

FIG. 19C is a cross-sectional view taken along line X9-X9 in FIG. 19A;

FIG. 20 is a schematic representation of the angioplasty enhancement device of FIG. 17A integrated into an angioplasty system in an Rx form of deployment; and

FIGS. 21A-21C are schematic representations of an axial cross-section illustrating three successive states in the deployment of the device of FIG. 17A within a blood vessel.

### DESCRIPTION OF THE PREFERRED EMBODIMENTS

The present invention is a device for percutaneous deployment to remove material from an internal surface of a blood vessel, and corresponding methods.

The principles and operation of devices and methods according to the present invention may be better understood with reference to the drawings and the accompanying description.

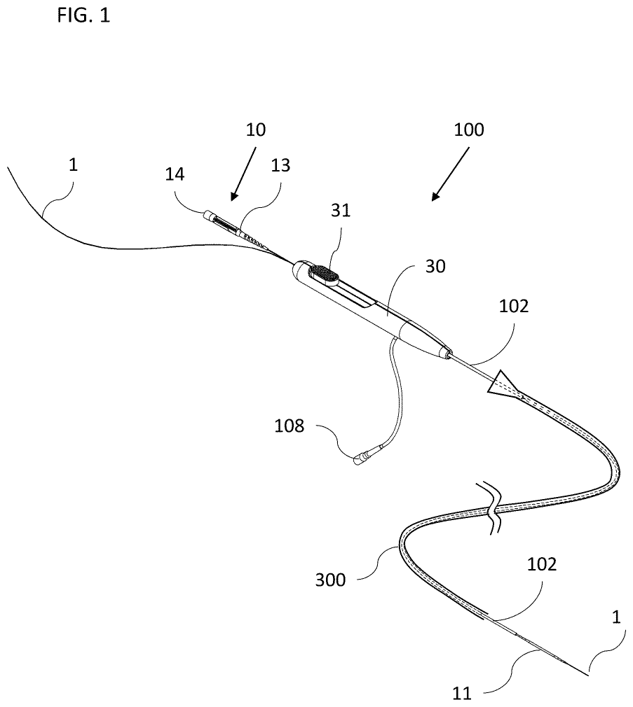

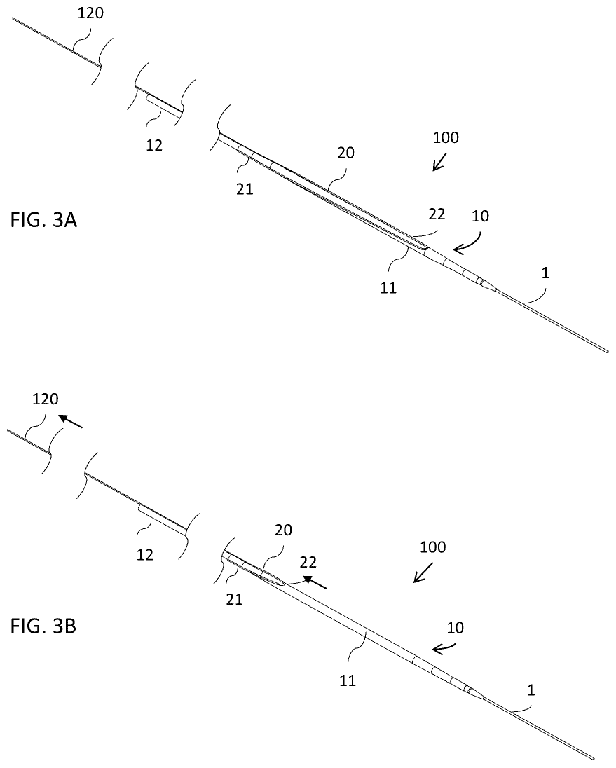

Referring now to the drawings, FIGS. 1-21C illustrate the structure and operation of a device, generally designated 100, constructed and operative according to an embodiment of the present invention, for percutaneous deployment to remove material from an internal surface of a blood vessel within a body of a human. In general terms, the device includes a deployment catheter 102 for percutaneous deployment within the blood vessel, and an inflatable balloon 11 configured for deployment from catheter 102. A loop 20 of flexible material (visible in the enlarged distal views of FIGS. 3A-8C) is displaceable across an external surface of balloon 11 for deployment with the balloon. The loop is preferably implemented as a loop of wire, referred to as a “scraping wire.” At least one side of loop 20 is connected via a displaceable element 120 extending along the catheter so that displacement of element 120 outside the body controls displacement of loop 20 relative to the balloon 11. Element 120 may be an extension of one or both sides of loop 20, or may be a separate element attached to the loop. Loop 20 is displaceable between a retracted, proximal position, shown in FIGS. 3B, 4A and 5A, and an extended, distal position, shown in FIGS. 3A, 4B, 5B and 6A, in which the loop 20 at least partially overlaps a length of the balloon 11.

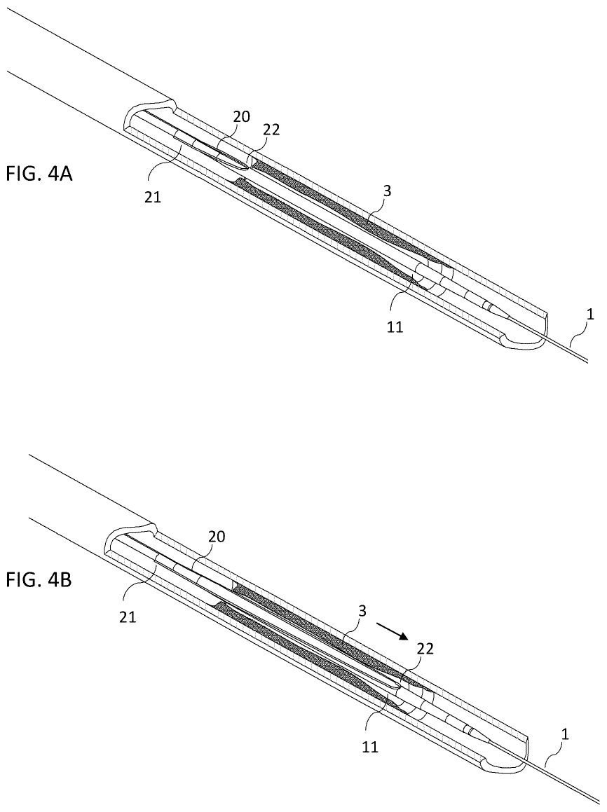

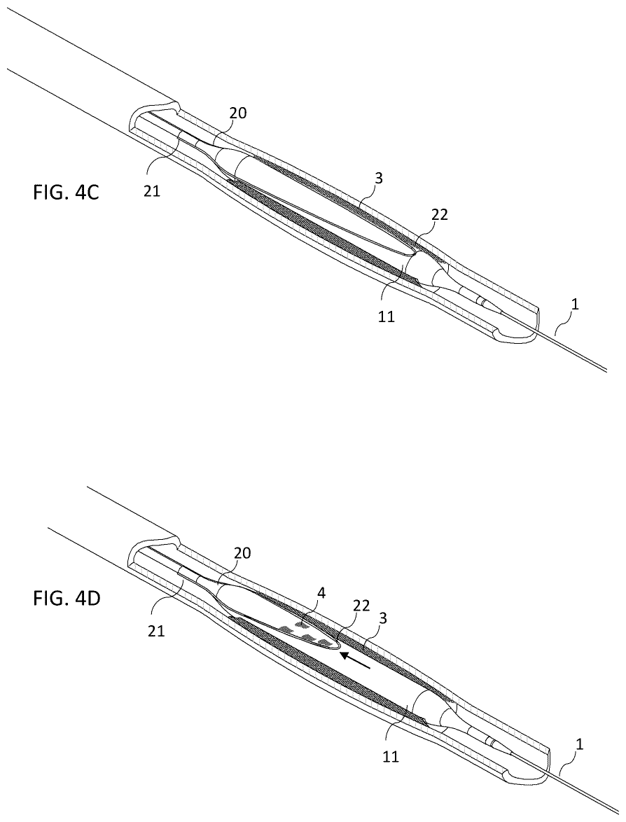

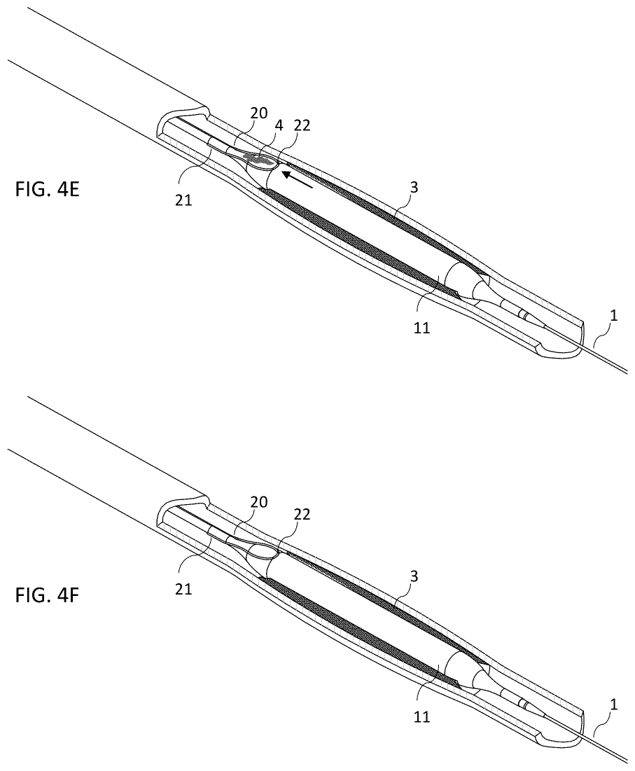

Balloon 11 and loop 20 are configured such that, when the balloon is inflated while the loop is in its distal position, the loop of flexible material is pressed against the internal surface of the blood vessel (FIGS. 4C, 5C and 6B). Subsequent pulling of the loop (FIGS. 4D, 4E, 5D, 5E and 6C) is effective to scrape material (typically plaque) from the internal surface of the blood vessel and to gather the material within the loop towards a proximal end of the balloon. The collected material is then aspirated, prior to deflation of the balloon. After at least partial deflation of the balloon 11, loop 20 can be pushed so as to return the loop to a distal position, allowing the scraping and aspiration process to be repeated as needed.

Catheter 102 may be implemented either as an over-the-wire (OTW) or rapid exchange (Rx) catheter, which facilitates removing plaque and other deposits from a vessel wall where the removing operation is safely done without letting the debris drift and clog capillary vessels downstream.

It will be appreciated that the invention as defined herein provides a low-risk approach for removing atheroma and/or other forms of plaque from vessels, employing an expandable balloon covered by cord layers, used for longitudinally fracturing, scraping and removing of the plaque from fibrous and calcified lesions.

The longitudinal fracture of the calcified deposits is achieved by the entire expanding force of the balloon. That force is focused on the wires that are interposed between the vessel wall and the balloon. This typically reduces barotrauma to the entire lesion and may thus provide one or more of the following advantages: reduces the extent of dissection; lowers the risk of rupture; reduces plaque shift; decreases vessel elastic recoil; and potentially reduces inflammation. Since the degree of vascular injury and dissection is known to be a correlator of restenosis, there is a significant potential in improved outcomes.

Optionally, the device may be used with the loop withdrawn, like a traditional, low rigidity, high crossable angioplasty balloon. Then, in the event that plaque fracturing or scraping is desired, the balloon is deflated and the loop is advanced distally over the balloon. The balloon is then re-inflated so that the wire causes the plaque to fracture longitudinally. Then, by retracting the wire, the plaque is scraped and gently removed backward to the proximal side of the balloon.

In certain embodiments, one end of the scraping wire 20 is fixed to the proximal end of inflatable balloon 11, or proximal thereto, while the other side of the loop extends back along the catheter as element 120 for bidirectional displacement (pulling and pushing) to retract and re-extend the loop relative to the balloon. In alternative embodiments, the loop of the scraping wire 20 is a closed loop, formed for example by fixing an end of the wire to a proximal region of the wire, so that retraction of element 120 passing along the catheter simultaneously retracts both sides of the U-shaped loop portion adjacent to the balloon.

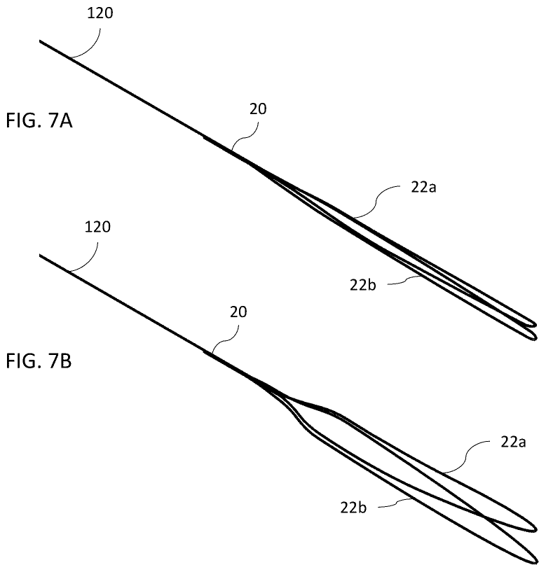

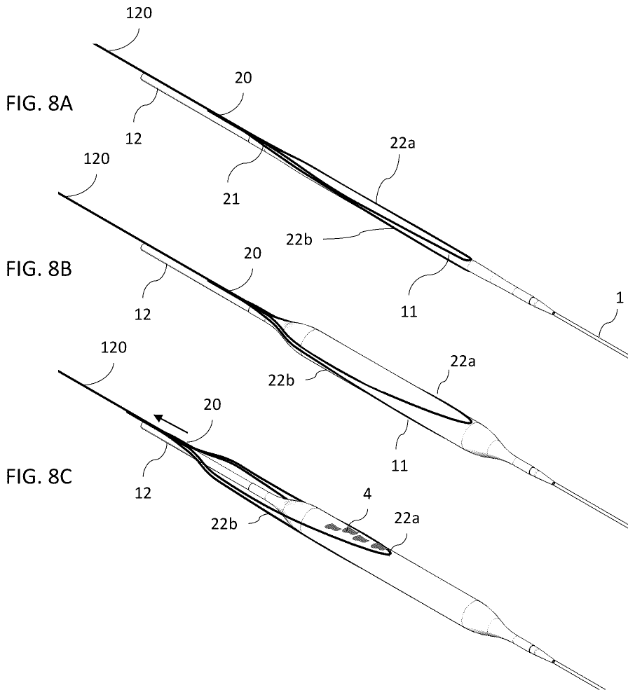

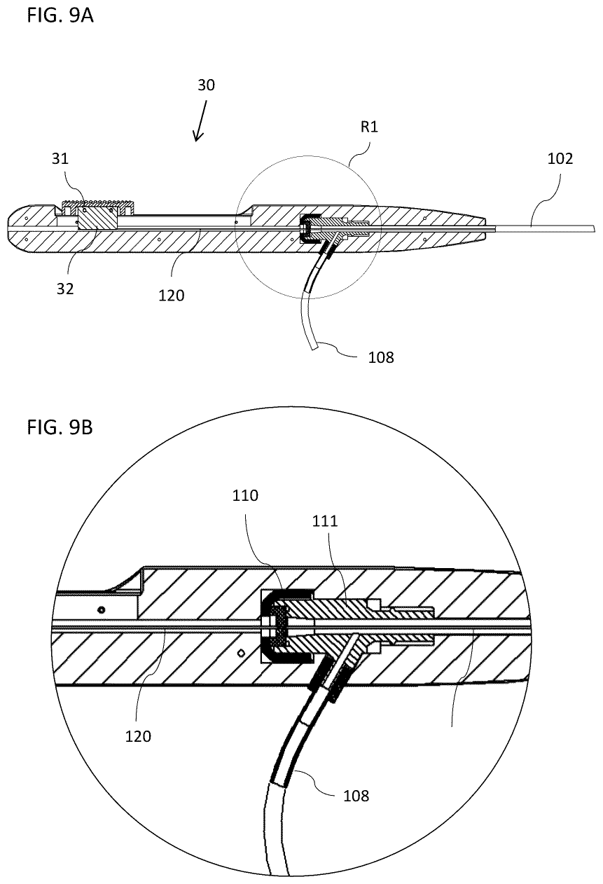

In a preferred implementation, there are at least two loops of wires, so that both sides of the balloon can be scraped simultaneously, as illustrated below in FIGS. 7A-8C. A plurality of loops provide more efficient and symmetric fracturing, scoring and scraping operations. The two or more loops are preferably connected by welding, soldering, adhering, crimping or any other suitable technique, so that they are displaced together via a single displacement element 120.

The one or more loops of the scraping wire are preferably made from a highly elastic material such us, for example, super elastic nitinol, tungsten or spring tempered stainless steel. The dimension of the wire can vary, for example, from 0.05 mm to 0.5 mm, chosen according to the dimensions of the balloon, the material properties and the mechanical properties required.

The wire is most preferably pre-formed into a U-shape such that the shape required when the balloon is inflated corresponds to the unstressed form of the wire. This could be done by standard methods of cold or hot wire forming technique. This preforming provides a resilient bias which facilitates returning the wire to its initial position for repetition of the scraping process. The loop is preferably elastically compressed into a lower-diameter form within the catheter.

Referring now in more detail to the drawings, FIG. 1 shows an overview of a typical but non-limiting application of the present invention, where the scraper balloon (SB) catheter device 100 is used together with a standard OTW or Rx balloon catheter 10 terminating in a balloon 11, which is deployed over a standard guidewire 1. These elements are all introduced at least part of the way to the target region along a minimally-invasive access route via a conventional guiding catheter 300, as is known in the art. Catheter 102 is preferably associated with a handle 30 which includes a slider 31 or other controller for controlling displacement of the displacer element (not visible in FIG. 1). Where aspiration is performed via catheter 102, an aspiration port 108 is also provided.

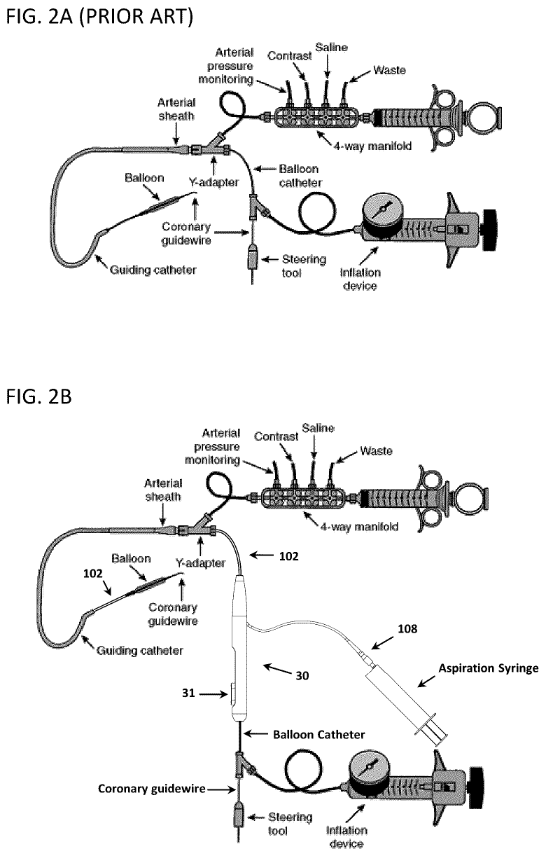

A non-limiting example of the integration of device 100 into a conventional balloon catheter setup is explained with reference to FIGS. 2A and 2B. FIG. 2A illustrates schematically an unmodified conventional system, with the various components labeled. A guiding catheter is introduced to near the target location, and a guidewire is inserted through the guiding catheter so as to extend beyond the guiding catheter to the target location. A balloon catheter is inserted over the guidewire (in this case OTW), and is connected to an inflation device. The guiding catheter may be connected to various additional inputs and outputs, such as via the 4-way manifold illustrated here.

FIG. 2B illustrates how the system of FIG. 2A may be modified to employ the device of the present invention. The catheter 102 is inserted via the guiding catheter and over the guidewire, and the balloon catheter is introduced through catheter 102. Various options for the arrangement of lumens within catheter 102 will be discussed in detail below.

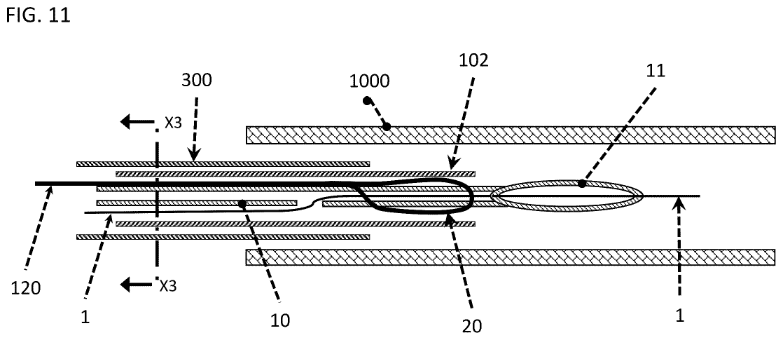

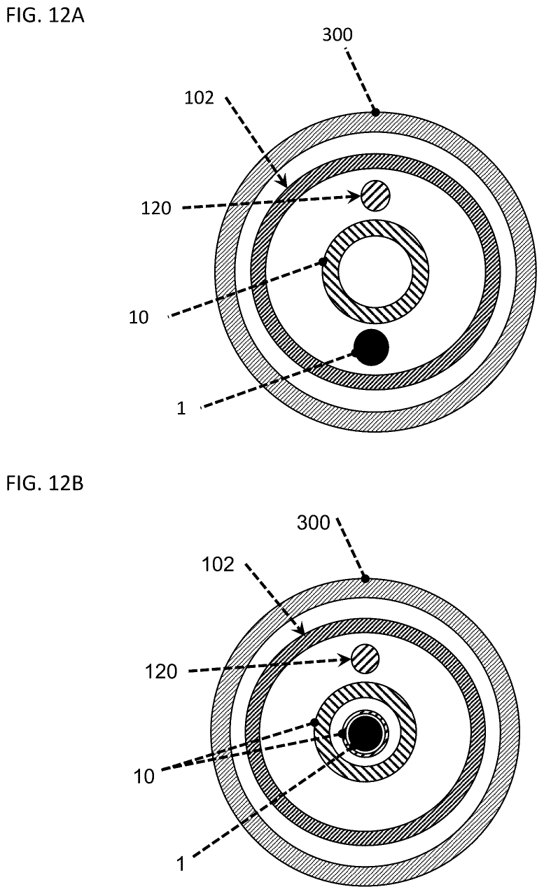

Specifically, FIGS. 11 and 12A illustrate schematically one preferred but non-limiting implementation of Scraper Balloon (SB) Catheter device 100 which employs a standard off-the-shelf rapid exchange (Rx) which may be according to the state of the art in design of a Percutaneous Transluminal Coronary Angioplasty (PTCA) balloons, and selected according to the preferences of the practitioner. Thus, there is shown a guiding catheter 300 inserted along a coronary blood vessel 1000, that typically stops short of the desired target location, and a guidewire 1, inserted along the guiding catheter and extending beyond the guiding catheter so as to cross the treatment region. Catheter 102 is in this case implemented as a catheter, sized to fit along guiding catheter 300, with a relatively large single lumen that accommodates balloon catheter 10, guidewire 1 and wire loop 20 or displacement element 120 (depending on the axial location), as shown in the cross-sectional view of FIG. 12A. At the location of the cross-section, for an Rx balloon catheter, guidewire 1 is external to the shaft of balloon catheter 10. In a variant implementation using an OTW balloon catheter, the guidewire is internal to the shaft of the balloon catheter, as illustrated in FIG. 12B.

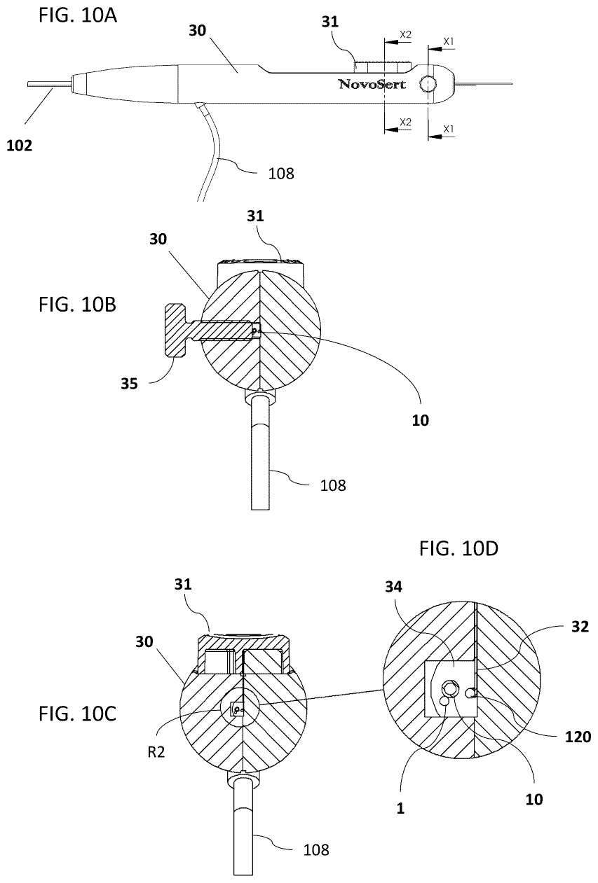

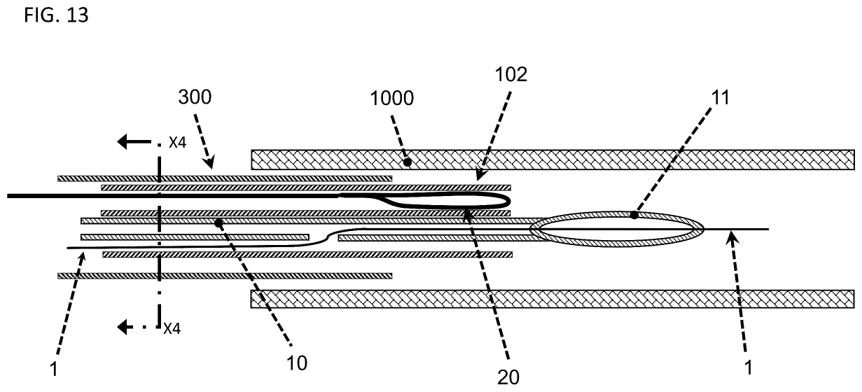

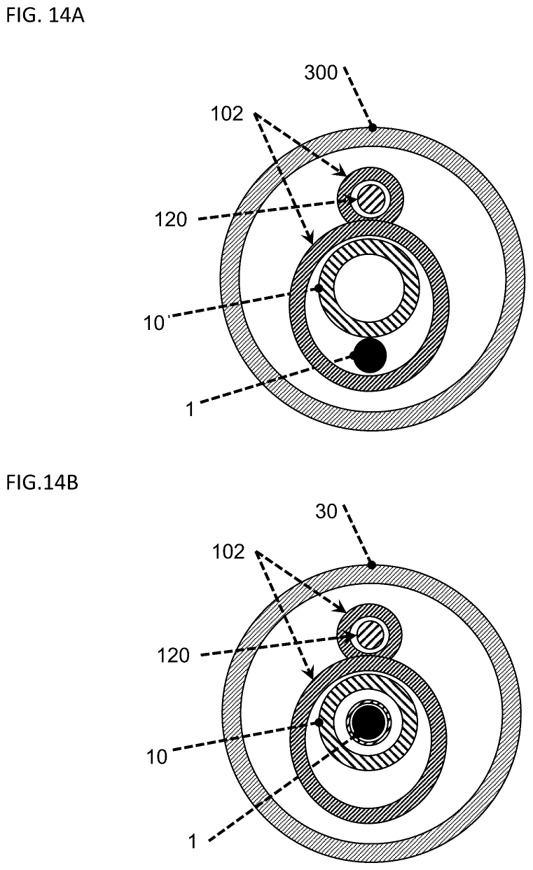

FIGS. 13 and 14A illustrate an implementation which is essentially similar to that of FIGS. 11 and 12A, but in which catheter 102 provides two distinct lumens, one for the balloon catheter and guidewire, and one for wire loop 20 or displacement element 120 (depending on the axial location). Here too, guidewire 1 is shown external to the balloon catheter shaft at the location of the cross-section for an Rx balloon catheter (FIG. 14A), but may alternatively employ an OTW balloon catheter, in which case guidewire 1 is internal to the shaft of the balloon catheter 10, as illustrated in FIG. 14B.

Where a standard balloon catheter, separate from catheter 102, is used, the balloon catheter should be clamped so as to prevent longitudinal drift of the balloon catheter relative to catheter 102 when force is applied to advance and retract the loop of wire 20. This may be achieved simply and effectively by employing a clamping screw 112 (FIGS. 10A and 10B) to manually clamp the position of the balloon catheter at handle 30.

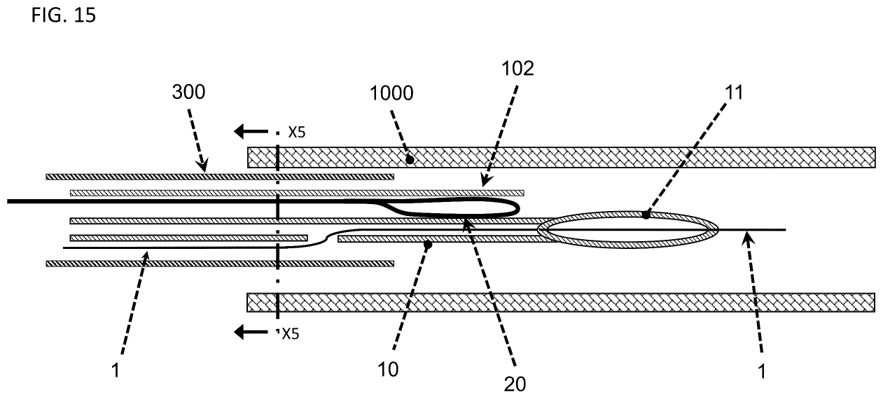

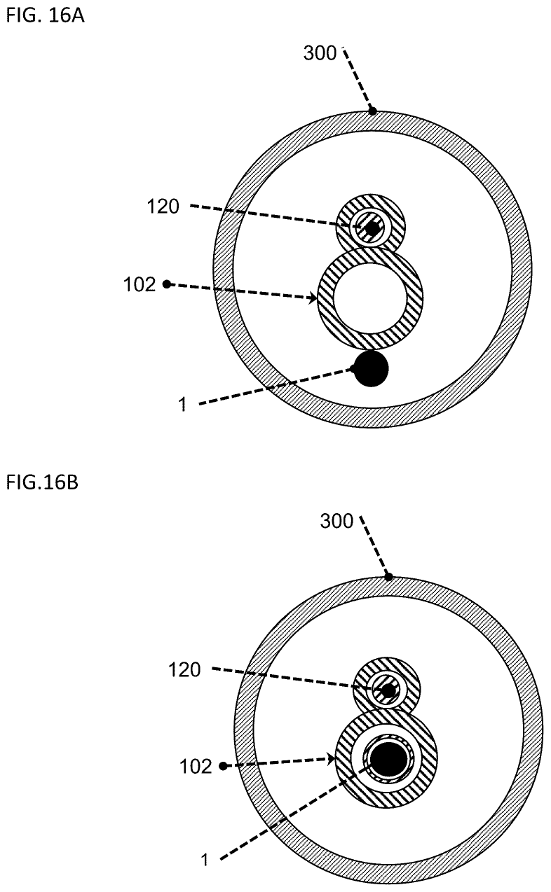

In an alternative set of implementations, illustrated schematically in FIGS. 15-16B, catheter 102 may be implemented with an integrated balloon 11. In this case, catheter 102 has at least two distinct lumens, one for inflation of the balloon and one for accommodating wire loop 20 or displacement element 120 (depending on the axial location), and typically also a third lumen for at least part of the length of the catheter for accommodating the guidewire 1. For an Rx implementation, the guidewire may be external to catheter 102 for most of its length (FIG. 16A) whereas, for an OTW implementation, a guidewire lumen is preferably provided along the entire length of catheter 102. This may be concentrically within the inflation lumen, as shown in FIG. 16B, as is commonly implemented for balloon catheters. Alternatively, side-by-side lumens, for example, in a triangular configuration (not shown) may be preferred for simplicity of manufacture.

Turning now back to FIGS. 3A-4F, these illustrate an implementation of scraping wire 20 in which one end 21 of the wire is fixed to a proximal end of the balloon 11. As illustrated in FIGS. 3A and 4B, scraping wire 20 extends along the balloon to its distal side where it turns (region 22) to form a U shape that extends along the other side of balloon 11 and along balloon catheter shaft 12, forming displaceable element 120 for controlling the loop from the proximal end of the device.

The scraping wire is preferably made of a highly elastic material such us, for example, super elastic nitinol, tungsten or spring-tempered stainless steel. The dimensions of the wire are typically between 0.05 mm to 0.5 mm diameter, according to the dimensions of the balloon 11 and the required mechanical properties. In a typically example, the loops are made of 0.1 mm steel or nitinol wires. Note that a standard 0.014″-compatible PTCA balloon typically has a crossing diameter of about 0.9-1.2 mm when deflated. A guidewire of 0.014″ diameter corresponds to about 0.36 mm diameter. Thus, by way of one non-limiting example, the present invention may be used with a 6 fr or 7 fr guiding catheter, with internal diameters of 1.78 or 2.06 mm, respectively. In such an example, the SB catheter device 100 may be implemented as a 5 fr catheter with an outer diameter of 1.65 mm and in internal diameter of 1.42 mm. This renders the arrangement compatible with almost any off-the-shelf PTCA balloon catheter.

The state of FIG. 4C preferably corresponds to an unstressed state of scraping wire 20. By pulling the proximal end of the wire, the U-bend (“loop”) of the wire is rolled and retracts to a proximal position illustrated in FIGS. 3B and 4D-4F. Pushing the wire back will roll and advance the U bend to its natural position of FIG. 4C.

Retracting and advancing the proximal end 23 of scraping wire 20 in relation to the shaft 11 of the balloon catheter 10 is typically performed manually, for example, using a push-pull handle mechanism such as that described below with reference to FIGS. 9A-10D.

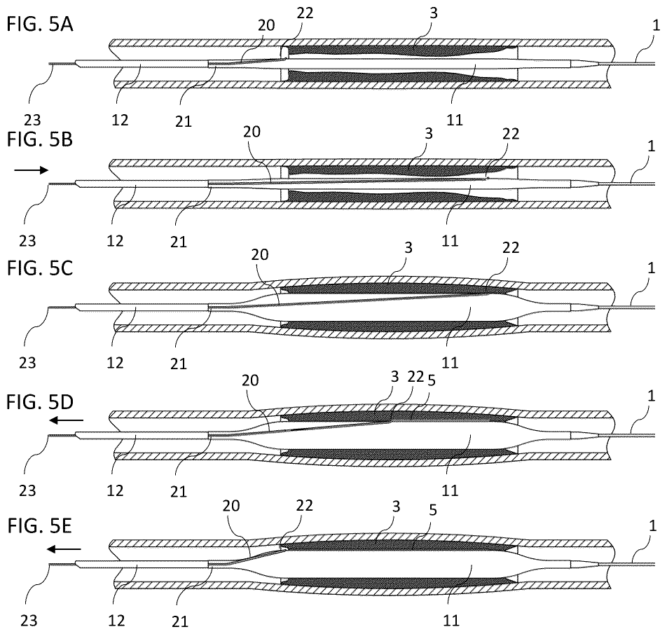

A typical sequence of operations for using the SB Catheter for scraping plaque and other deposits from the internal walls of the vessel is illustrated in the sequences of FIGS. 4A-4F, 5A-5E and 6A-6C. The sequence of operations illustrated in those figures includes:
1. Delivering of the SB Catheter 100 over the guidewire 1 to the occluded area 3 (FIGS. 4A and 5A). Optionally, in this state, the practitioner may choose to operate the balloon in a conventional manner, without the scraping wire (which is located in a proximal position, in non-overlapping or minimally-overlapping relation to the balloon), either as a preparatory step to deployment of the scraping wire, or as an alternative thereto. If/when the practitioner decides that the scraping operation should be performed, he or she proceeds to the following steps.
2. Advancing the scraping wire 20 to a distal position where its U-bend 22 is located at or near the distal end of balloon 11 (FIGS. 4B, 5B and 6A). (As an alternative, if the option to deploy the balloon without the scraping wire is not required, the wire may be inserted together with the balloon already in this distal position.)
3. Inflating the balloon 11 so that the balloon presses scraping wire 20 against the deposits/sediments on the internal wall 3 of the vessel (FIGS. 4C, 5C and 6B).
4. Retracting the scraping wire 20 along the vessel wall 3, and thereby scraping plaque sediments 4 from the vessel wall. The plaque is scraped by the retracting wire to a position proximally to the balloon (FIGS. 4D, 4E, 5D, 5E and 6C). The scraping wire and the balloon prevent the debris 4 from drifting with the blood flow to clog a smaller capillary vessel downstream. The debris 4 is preferably flushed out from the vessel, either using a dedicated catheter or, in certain preferred embodiments, aspiration of the debris 4 may be performed via a lumen of catheter 102.
5. Deflating the balloon then returns the system to the initial state described in FIGS. 4A and 5A.
6. The scraping wire loop can then be advanced again to its distal position of FIGS. 4B and 5B, to allow repetition of the scraping procedure.

Although presented above as a single “inflated” state, in certain particularly preferred implementations of the invention, two different levels of inflation pressure may be used. A typical inflation pressure for a PTCA balloon to achieve rupturing of plaque is preferably in excess of 5 atm., and typically in the region of 6 atm. This pressure is preferably also used in the context of the present invention, so that the full plaque-rupturing effect of the balloon is achieved, further enhanced by the presence of the sides of the one or more loops of wire of the present invention, which provide local concentration of forces on the plaque, similar to the effect of a cutting balloon. At these high pressures, the wire loop is typically pinched between the balloon and the plaque deposits so that the wire cannot be moved my manual force applied to the slider. After the initial plaque-rupturing expansion, the inflation pressure is therefore preferably reduced, preferably to a pressure of no more than about 2 atm., and typically between 1-2 atm., in order to maintain effective contact pressure of the loop against the lesion while allowing manual displacement of the loop to perform a desired scraping effect. This intermediate pressure is also sufficient to maintain sealing of the blood vessel so that debris scraped proximally from the lesion by the motion of the wire does not escape distally before it is aspirated.

In certain applications, a practitioner may choose to employ the loops of the present invention for the cutting-balloon effect as described above, without any step of scraping. The device of the present invention provides profound advantages over conventional cutting balloons in that the wires provide a similar effect while using a conventional small-diameter flexible PTCA balloon, thereby greatly enhancing the range of scenarios in which a lesion can be successfully crossed by the balloon and, after insertion of the wires, a cutting-balloon effect can be achieved.

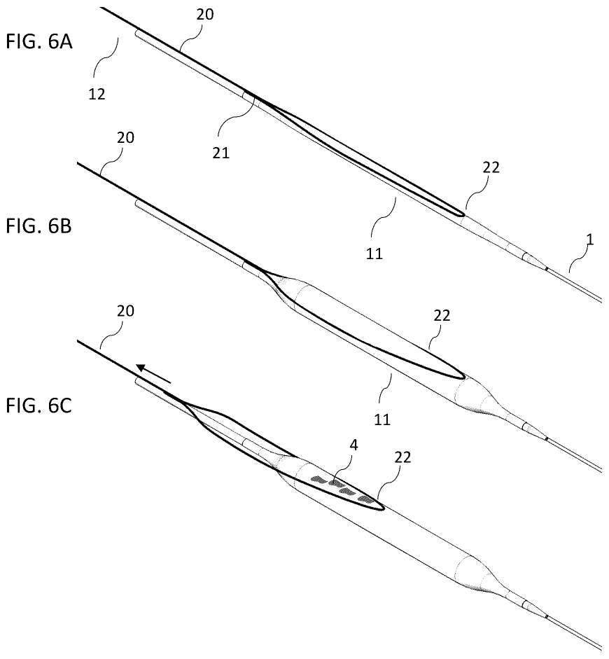

In the non-limiting example illustrated in FIGS. 4A-5E, the scraping loop is illustrated as being an “open loop” of which one end 21 is deployed in a fixed position relative to the balloon 11, as detailed above. FIGS. 6A-6C illustrate a further non-limiting example in which the entirety of the loop 20 is displaceable relative to balloon 11. In this case, the scraping wire 20 is preferably formed into a closed loop by attaching the end 21 of the wire to the proximal side of the loop by welding, soldering, crimping, or any other suitable form of connection. FIG. 6A illustrates the distal tip of the loop 22 advanced to a position next to the distal end of balloon 11. FIG. 6B illustrates the state after inflation of balloon 11 so that loop 20 is pressed against the wall of the occluded area, and FIG. 6C shows loop 20 being retracted while pressed against the wall of the blood vessel. The structure and function of this closed-loop implementation is thus fully analogous to that described above with reference to FIGS. 4A-5E.

The above sequence can be repeated as required, to perform repeated scraping and aspiration of material from the wall of the vessel. Optionally, and advantageously, the loop, and typically the entirety of the SB catheter, is rotated about a longitudinal axis of the inflatable balloon, typically before advancing the wire loop, either between each successive scraping action or intermittently after a number of scraping actions in a given position. As a result, a first scraping step scrapes material from a first region of the internal surface of the blood vessel, and a repetition of the scraping step scrapes material from a second non-identical region of the internal surface of the blood vessel. The successive regions may be partially overlapping, and may be part of an angular progression of regions which progressively extend the region scraped until the entire internal surface of the blood vessel has been scraped.

A further approach to enhancing the angular coverage of the scraping wire is to provide two or more displaceable loops spaced around a periphery of the balloon. This option is illustrated in FIGS. 7A and 7B, which show a scraping wire 20 with two loops 22a and 22b, shown in a compressed state and an expanded state, respectively. Both loops are preferably displaced simultaneously by a common displacement element 120. FIGS. 8A-8C parallel FIGS. 6A-6C but illustrating the stages of operation of the two-loop implementation of FIGS. 7A and 7B. The operation is fully analogous to the single-loop embodiment described above, and will be understood by reference to the description above. The use of multiple loops together with rotation allows the internal surface of the blood vessel to be covered by a smaller number of axial rotations, since two opposing regions are scraped for each given position.

According to certain preferred implementations, displacement of loop 20 is controlled by an actuator, such as a manually-operable slider 31, associated with a handle 30, which allows displacement of displaceable element 120 relative to balloon catheter 10, and hence controls advancing and retracting loop 20 relative to balloon 11.

One non-limiting exemplary implementation of a handle mechanism for advancing and retracting the scraping wire 20 is illustrated in FIGS. 9A-10D. The handle 30 has a manually-operable slider 31 fixed via a slider plate 32 to the proximal end of the displacement element 120, which may be a proximal extension of scraping the wire forming loop 20. The slider is slidably received within the body of handle 30 that is fixed to deployment catheter 102. Where an interchangeable PTCA balloon catheter 10 is used, the balloon catheter is preferably inserted along a channel 34 which passes through handle 30 and feeds into a corresponding lumen of deployment catheter 102. In order to fix the longitudinal position of the balloon catheter 10 in order to allow displacement of loop 20 relative to the balloon catheter, the axial position of balloon catheter 10 within channel 34 is preferably fixed after insertion by tightening a clamping screw 35 (FIG. 10B). The location of clamping screw 35 is preferably proximal to slider 31, or otherwise separated from displacement element 120, so that the clamping screw acts only on the shaft of balloon catheter 10. The force applied by the hand-tightened clamping screw does not crush the balloon catheter shaft, which is typically made of strong materials, such as stainless-steel. The guidewire is preferably also clamped by the same clamping mechanism, or by a separate clamping mechanism (not shown).

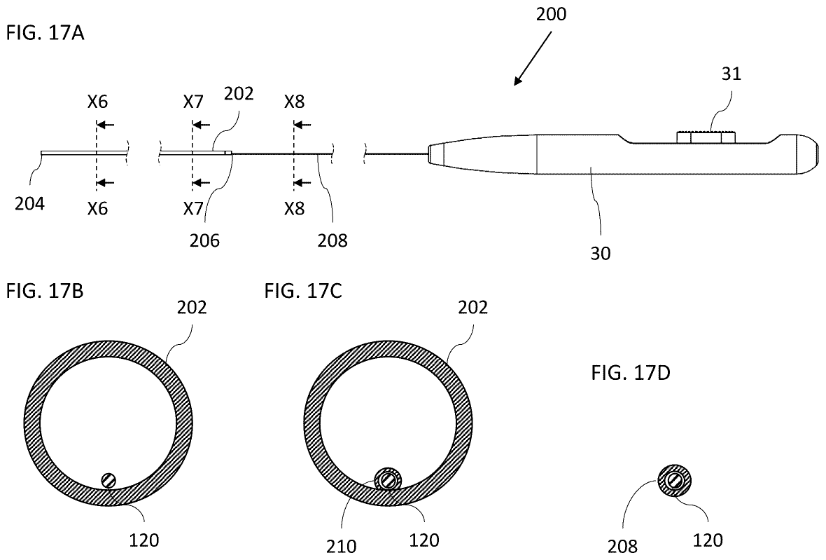

A hub 13 (FIGS. 1 and 17) provides a connection to the multi-lumen shaft of balloon catheter 10 with a filling port 14 accessible at the proximal side of handle 30 for inflating and deflating balloon 11 via an inflation lumen.

Where catheter 102 provides an aspiration function for removing scraped debris, the relevant lumen is preferably connected to an aspiration port 108 via a hemostatic valve 110 deployed in a hemostatic valve housing 111 within handle 30 (FIG. 9B). Optionally, a dedicated lumen for aspiration may be provided. Alternatively, the aspiration function may not require a dedicated lumen, and can instead be performed via either the lumen that accommodates the balloon catheter, or that which accommodates the displacement element 120 (or the common lumen, if both pass within a shared lumen). The lumen to be used for aspiration should be designed to have sufficient open cross-section to allow passage of the expected size of debris particles. Optionally, a lumen can also be provided (optionally shared with another purpose) for introduction of saline during aspiration for flushing out and removing dislodged plaque or other deposits scraped from the vessel internal surface. During this flushing and aspiration process, the balloon 11 preferably remains inflated, thereby providing an efficient barrier against drifting of the scraped debris downstream. Additionally, the U-shape of the wire together with the directional scraping helps to ensure that the scraped debris is removed proximally to the balloon, where a flushing and suction system can remove it from the vessel. As mentioned, aspiration does not necessarily have to be performed via catheter 102, and may instead be performed via a separate dedicated aspiration catheter that is introduced for this purpose.

The handle structure illustrated here is suitable for fully-manual operation of the scraping device. In some cases, it may be desired to partially or fully automate the sequence of operations performed by the scraping wire and the balloon. In that case, the operations which are automated typically include: advancing the scraping wire; inflating the balloon; and retracting the scraping wire. Optionally, steps of actuating aspiration; deflating the balloon; and optionally also rotating the device, can also be automated.

Such automation may be implemented by connecting the handle to a mechanism that provides controlled reciprocating movement of the slider synchronized with controlled operation of a pressure pump that inflates and deflates the balloon according to the position of the slider; such that this device catheter could operate repeatedly in an automatic manner to remove plaque from a vessel walls, preferably also synchronized with flushing/aspiration steps. Alternatively, in another preferred embodiment, semi-automatic operation may be provided, for example, where inflation (and optionally also aspiration and deflation) of the balloon is actuated automatically in response to manually controlled movement of the scraping wire. The various components (e.g., position sensor, pressure sensor, a motion actuator, pump or switchable manifold connected to suction and saline, control logic circuitry etc.) and their arrangement to achieve the automatic or semi-automatic operation of the system, will be self-evident to a person ordinarily skilled in the art, and will not be described herein in detail.

Suitable materials for the scraping wire are essentially any surgical grade materials with sufficient elastic properties to be collapsible within the catheter, to return to the desired loop form when advanced beyond the catheter, and to be sufficiently self-supporting to allow the loop to be advanced alongside the (collapsed) balloon along the vessel. Examples of suitable materials include but are not limited to: stainless steel, nitinol and tungsten alloys, as well as various non-metallic threads or filaments, such as Kevlar or carbon fiber filaments.

The scraping wire is advantageously plated, partially or entirely, with an Au, Pt or Ta radiopaque layer, to facilitate real time fluoroscopic imaging. Additionally, or alternatively, the scraping wire may be coated with abrasive fine (e.g., micron size) diamond powder that helps in scraping the plaque.

According to a further alternative, the scraping wire may be composed of a coil threaded over a wire, optionally where the coil is made of a radiopaque thin wire. The coil shape helps in scraping the plaque. An example of such a configuration for the scraping wire would be a coil made of a thin (e.g., Ø0.01 to Ø0.07 mm) tungsten wire having a coil outer diameter of Ø0.15 to Ø0.35 mm threaded over a nitinol or stainless-steel wire having the same elastic properties as the scraping wires listed above.

The preformed shape of the scraping wire loop (or loops) to which the wire tends to return when extended beyond the catheter tip preferably has a rounded distal tip with a radius of curvature no more than about 1 mm, and preferably no more than about half a millimeter (roughly 1 mm diameter), thereby facilitating introduction of the loop into a catheter, and providing a narrow but smooth tip which is able to be pushed distally so as to find its way through a small opening in an occlusion. A medial region of the loop is preferably preformed so as to be biased to assume an open-loop state with a width between the two sides of the loop that is at least twice, and preferably at least three times, the diameter of the distal tip. In absolute terms, the unstressed width of the medial part of the loop for a significant range of applications is preferably within the range of 2.5-4 millimeters.

It is a particular feature of an aspect of the present invention that the scraping loop (or loops) are selectively deployable, and can be selectively advanced next to the uninflated balloon during a procedure. In other words, the practitioner may choose to deploy and expand a balloon at the region of an occlusion while the scraping wire remains retracted, partially or fully within its catheter, and inoperative (in the position shown in FIG. 4A). If the procedure is successful without deploying the scraping wire, the scraping wire will not be deployed at all. If, during the procedure, the practitioner decides to deploy the scraping wire, he deflates the balloon (if previously inflated), and advances the loop(s) next to the balloon so as to traverse the partial occlusion, to the position of FIG. 4B, and then proceeds according to the sequence described above with reference to FIGS. 4B-4F and FIGS. 5B-5E. The sequence can be repeated, as also mentioned above.

It is a particular feature of a further aspect of the present invention that the scraper device is provided as an add-on tool that can be used with substantially any off-the-shelf angioplasty balloon, chosen according to the preference of the practitioner and according to the case-specific needs, including balloons of differing expanded diameters, and both elastomeric and non-stretching (fixed diameter) balloons, as well as drug eluting balloons or any other balloon with particular features or properties preferred by the practitioner. For this purpose, the scraper device is preferably provided together with its catheter, and with an open lumen for insertion over a guidewire followed by insertion of an over-the-wire (OTW) or Rapid Exchange (Rx) balloon catheter. The catheter preferably also provides an aspiration lumen (optionally shared with one of the other lumen functions) for applying suction, via a syringe or another source of suction, to aspirate debris that is dragged to the proximal side of the balloon-blocked vessel by the closed loop of the scraping wire.

It should be noted that the above embodiments, as well as a further embodiment described below with reference to FIGS. 17A-21C, may provide the additional functionality of a “Guide Extension Catheter.” A Guide Extension Catheter is a smaller gauge guiding catheter which is placed within a larger guiding catheter in order to provide added support for the crossing of lesions or for the distal delivery of balloons and stents. This technique has been described in an article by Takahashi entitled “New Method to Increase a Backup Support of Six French Guiding Coronary Catheter,” published in Catheterization and Cardiovascular Interventions, 63:452-456 (2004).

In the embodiments presented above, the options of FIGS. 12A and 14A allow for the use of a standard rapid-exchange (RX) balloon catheter, but require the guidewire to be inserted along the SB catheter device 100 for its entire inserted length, either in an OTW deployment procedure (which requires use of a long guidewire), or by reversing the deployment sequence to use the catheter as a guide for insertion of the guidewire. The option of FIG. 16A allows for the entire device to be implemented as an RX device, but employs a built-in balloon. In such an implementation, the practitioner does not have the flexibility to choose an off-the-shelf balloon catheter of his or her preference.

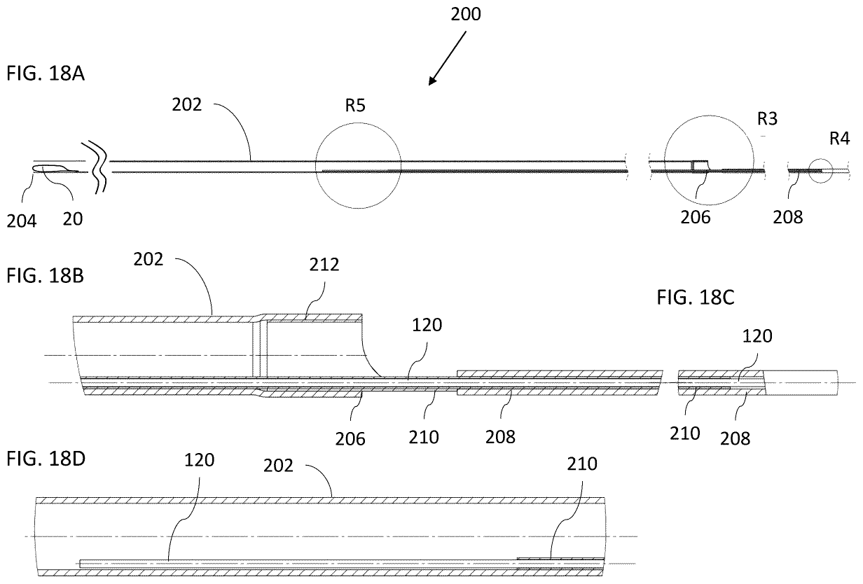

A further particularly preferred but non-limiting embodiment of the present invention, described herein with reference to FIGS. 17A-21C, combines the advantages of a catheter device 200 according to the teachings of the present invention which provides full rapid exchange (Rx) functionality while also allowing the use of a standard Rx PTCA balloon catheter 10 of the practitioner's choice. In this case, as seen in the overview of FIG. 17A, the deployment catheter is implemented as a rapid exchange guide extension catheter 202 (see also the cross-sections of FIGS. 17B and 17C) having a distal end 204 and proximal end 206. The proximal end is connected to a hollow shaft 208, along which actuating element (wire) 120 extends internally (see the cross-section of FIG. 17D). The proximal end of shaft 208 is connected to a handle 30 which includes a slider 31 or other input for controlling axial movement of actuating element 120, all as described above. As illustrated in FIG. 18A, wire loop 20 (or a pair of loops, not shown, such as loops 22a and 22b illustrated above) is located within the distal end 204 of the guide extension catheter 202, and extensible therefrom by advancing slider 31 and actuating element 120.

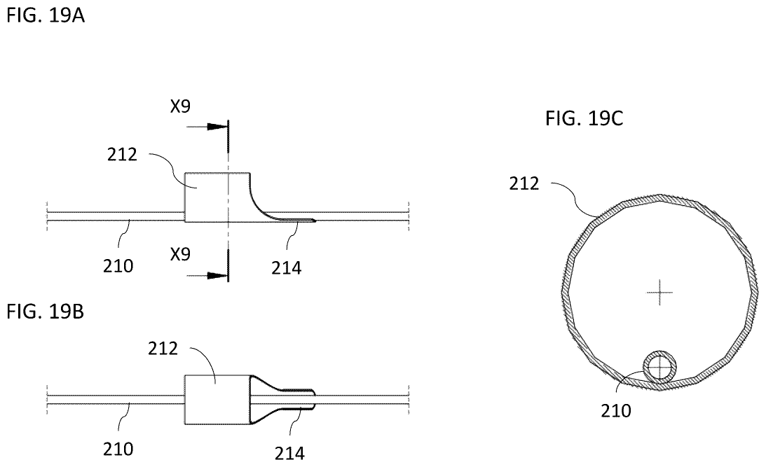

The transitions between different regions of a particularly preferred implementation of catheter device 200 are illustrated in FIGS. 18B-18D and 19A-19C. As best seen in FIGS. 18B and 18C, hollow shaft 208 has a relatively large diameter and wall thickness over a majority of its length from proximal handle 30 to provide good “pushability” to the catheter, and preferably transitions in a distal shaft portion 210 to a smaller diameter and wall-thickness to provide flexibility and trackability to the catheter and to provide enough space for delivering an off-the-shelf PTCA balloon catheter through the extension guiding catheter. Both parts of the shaft are preferably implemented as “hypotubes” (small diameter metal tubes, similar to hypodermic needle tubes, for medical applications), preferably made out of stainless-steel or super elastic Nitinol alloy. The shafts are joined together by soldering, welding, adhesives, pressure-fitting or any other appropriate technique. Guide extension catheter 202 is preferably made of elastomeric materials or of a combination of polymer and reinforcing elements such as braided metallic coil or mesh.

As illustrated in FIG. 18B, the guide extension catheter 202 is preferably connected to the distal shaft portion 210 by a connector 212 located a short distance beyond a distal end of the proximal portion of shaft 208. Connector 212 together with distal shaft portion 210 are shown in more detail in FIGS. 19A-19C.

As shown in FIGS. 19A-19C, connector 212 is preferably implemented as a hypotube, preferably also of stainless steel or super elastic Nitinol alloy, connected internally to distal shaft portion 210 by soldering, welding, or any other appropriate technique. In the preferred but non-limiting example illustrated here, connector 212 is formed with a tab 214 protruding in a proximal direction. Tab 214 may be conveniently formed by removing part of the circumference of the hypotube in that region. In this example, distal shaft portion 210 is joined to tab 214. Proximal end 206 of guide extension catheter 202 is seated over the outer diameter of connector 212, and is attached thereto either by elastic compression of the catheter wall and/or by use of a suitable adhesive (for example, epoxy adhesive).

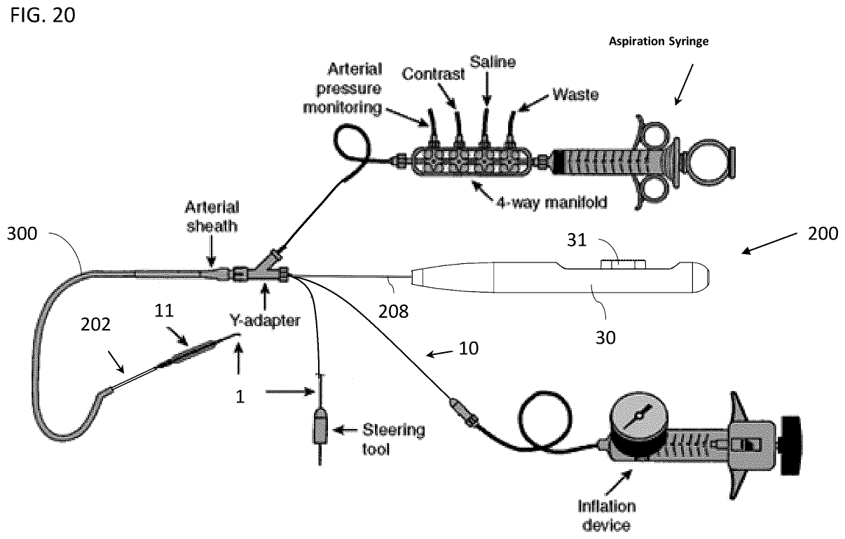

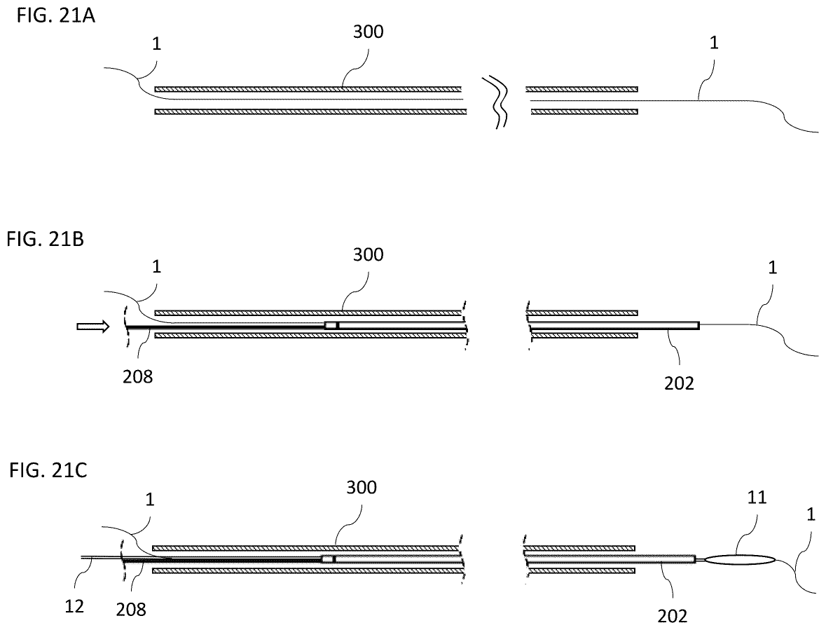

An overview of integration of catheter device 200 with a conventional setup for an interventional procedure employing an Rx PTCA balloon is illustrated schematically in FIG. 20. The corresponding stages of deployment are illustrated schematically in FIGS. 21A-21C. After placement of the guiding catheter 300 and introduction of guidewire 1 so as to cross the lesion (FIG. 21A, lesion not shown), catheter device 200 is preferably deployed with guide extension catheter 202 threaded over the proximal part of guidewire 1 and advanced along the guidewire in an Rx mode of deployment until it extends beyond the guiding catheter, providing guide extension catheter functionality (FIG. 21B). During subsequent introduction of an Rx balloon catheter, the balloon follows the guidewire and thus enters and passes through guide extension catheter 202, emerging at and crossing the lesion (FIG. 21C, lesion not shown). Within the majority of the length of guiding catheter 300, guidewire 1, balloon catheter shaft 12 and shaft 208 of catheter device 200 are in side-by-side relation within the guiding catheter lumen. Actuation of the loop 20 or loops so as to advance them over the surface of the uninflated balloon, for use as cutting elements and/or for scraping plaque from the lesion, then proceeds as described above with reference to FIGS. 3A-8C.

It should be noted that these drawings are not to scale, and that there is typically a very small clearance between the outer surface of guide extension catheter 202 and the internal surface of guiding catheter 300. By way of one typical but non-limiting example, if the guiding catheter is implemented as a 6 fr catheter with an internal lumen diameter of 1.78 mm and the guide extension catheter is implemented as a 5 fr catheter with an external diameter of 1.65 mm, the radial clearance between the two is roughly 65 microns. The combination of this small clearance together with a relatively long region of overlap generates a very high flow impedance for water-based fluids. As a result, suction applied to the lumen at the proximal end of the guiding catheter, for example, via the aspiration syringe of FIG. 20, is effective to perform aspiration of fluid and debris at the distal end of the guide extension catheter.

It will be appreciated that the above descriptions are intended only to serve as examples, and that many other embodiments are possible within the scope of the present invention as defined in the appended claims.

1. An angioplasty enhancement device for use with an angioplasty system including a guiding catheter, a guidewire inserted through the guiding catheter and extending beyond the guiding catheter to cross a lesion, and a balloon catheter insertable along the guidewire through the guiding catheter to reach the lesion, the angioplasty enhancement device comprising:
(a) an elongated shaft sized for insertion through the guiding catheter and to extend beyond the guiding catheter, at least a distal portion of said elongated shaft being formed as a guide extension catheter with at least one hollow lumen to accommodate the guidewire and the balloon catheter;
(b) a loop of flexible material displaceable between a proximal position located within said lumen of said guide extension catheter and a distal position extending beyond said guide extension catheter for overlying an external surface of the balloon catheter; and
(c) a displaceable element extending along said elongated shaft and associated with at least one side of said loop so that displacement of a part of said displaceable element from a proximal end of the guiding catheter controls displacement of said loop between said proximal position and said distal position,
such that, when the balloon catheter is inserted along the guidewire in a deflated state so as to extend across the lesion, pushing the displaceable element is effective to advance said loop from the proximal position over the external surface of the balloon catheter to the distal position so that subsequent inflation of the balloon catheter is effective to press the loop against the lesion.

2. The angioplasty enhancement device of claim 1, wherein a majority of a length of said elongated shaft proximal to said guide extension catheter is implemented as a tube sized for accommodating only said displaceable element while lying in side-by-side relation to the guidewire and the balloon catheter within the guiding catheter.

3. The angioplasty enhancement device of claim 1, wherein the loop is a wire loop formed with a U-shaped distal tip such that, when said wire loop is pressed by the balloon against the lesion, pulling of the wire loop is effective to scrape debris from the lesion and to gather the debris within the loop towards a proximal end of the balloon.

4. The angioplasty enhancement device of claim 1, wherein the loop is one of at least two loops spaced apart so as to advance around at least two respective regions of a periphery of the balloon catheter, said respective regions being on opposite sides of a central longitudinal axis of the balloon catheter.

5. The angioplasty enhancement device of claim 1, further comprising a handle associated with a proximal end of said elongated shaft, and wherein displacement of said loop is controlled by an actuator associated with the handle.

6-19. (canceled)

20. A device for percutaneous deployment to remove material from an internal surface of a blood vessel within a body of a human, the device comprising:
(a) a deployment catheter for percutaneous deployment within the blood vessel;
(b) an inflatable balloon configured for deployment from the catheter;
(c) and a displaceable loop of flexible material displaceable across an external surface of the balloon for deployment with the balloon, at least one side of the loop being connected via a displaceable element extending along the catheter so that displacement of a part of said displaceable element outside the body controls displacement of said loop relative to said balloon;
wherein the balloon and the loop of flexible material are configured such that, when the balloon is inflated, the loop of flexible material is pressed against the internal surface of the blood vessel so that pulling the at least one side of the loop is effective to scrape material from the internal surface of the blood vessel and to gather the material within the loop towards a proximal end of the balloon, and such that, when the balloon is at least partially deflated, pushing the at least one side of the loop is effective to return the loop to a distal position.

21. The device of claim 20, wherein the loop of flexible material is a wire loop.

22. The device of claim 20, wherein two ends of the loop are associated with the displaceable element so to form a closed loop.

23. The device of claim 20, wherein the loop is one of at least two displaceable loops for deployment across at least two respective regions of a periphery of the balloon, said respective regions being on opposite sides of a central longitudinal axis of the balloon.

24. The device of claim 20, wherein the deployment catheter comprises a handle, and wherein displacement of the loop is controlled by an actuator associated with the handle.

25. The device of claim 20, wherein at least a distal portion of the deployment catheter is implemented as a guide extension catheter having a lumen, and wherein the balloon is part of a balloon catheter inserted along the lumen of the guide extension catheter.

26. A method for removing material from an internal surface of a blood vessel within a body of a human, the method comprising the steps of:
(a) deploying within the blood vessel a percutaneously introduced inflatable balloon and a loop of flexible material adjacent to an external surface of the balloon, at least one side of the loop being displaceable relative to the external surface of the balloon from outside the body;
(b) inflating the balloon so as to press the loop of flexible material against the internal surface of the blood vessel;
(c) pulling the at least one side of the loop so as to scrape material from the internal surface of the blood vessel and to gather the material within the loop towards a proximal end of the balloon;
(d) at least partially deflating the balloon;
(e) pushing the at least one side of the loop so as to return the loop to a distal position; and
(f) repeating steps (b) and (c).

27. The method of claim 26, further comprising, subsequent to step (d), rotating at least the loop about a longitudinal axis of the inflatable balloon so that the first occurrence of step (c) scrapes material from a first region of the internal surface of the blood vessel, and the repetition of step (c) scrapes material from a second region of the internal surface of the blood vessel, said second region being non-identical to said first region.

28. The method of claim 26, further comprising, prior to step (d), aspirating the material scraped from the internal surface of the blood vessel from adjacent to the proximal end of the balloon.

29. The method of claim 26, wherein the loop is a closed loop displaceable in its entirety relative to the balloon.

30. The method of claim 26, wherein the loop is one of at least two displaceable loops spaced around a periphery of the balloon.

31. The method of claim 26, wherein the loop is associated with a catheter having a handle, and wherein displacement of the loop is controlled by an actuator associated with the handle, the catheter having a lumen configured for interchangeable insertion of a PTCA balloon.

32. The method of claim 26, wherein said inflating comprises:
(a) inflating the balloon to a pressure in excess of 5 atmospheres in order to break up a lesion; and
(b) lowering the inflation pressure to no more than 2 atmospheres prior to said step of pulling.
